(19) United States
(12) Patent Application Publication
(10) Pub. No.: US 2026/0111650 A1
(43) Pub. Date: Apr. 23, 2026

(54) METHOD AND APPARATUS FOR DETERMINING ONTOLOGY CODES UTILIZING A LARGE LANGUAGE MODEL ENGINE

(71) Applicant: Emtelligent Software Ltd., Vancouver (CA)

(72) Inventors: Anoop Dipesh SARKAR, Vancouver (CA); Timothy William O'CONNELL, Vancouver (CA)

(21) Appl. No.: 19/326,638

(22) Filed: Sep. 11, 2025

Related U.S. Application Data
(60) Provisional application No. 63/709,211, filed on Oct. 18, 2024.

(51) Int. Cl. G06F 40/14; G06F 40/279; G06F 40/40; G16H 15/00

(52) U.S. Cl. CPC G06F 40/14; G06F 40/279; G06F 40/40; G16H 15/00

(57) ABSTRACT

A method and apparatus is provided that includes identifying, using context-specific natural language processor (NLP), an ontology code associated with at least a portion of text from a document, determining a root code associated with the identified ontology code, obtaining, from a database, child codes associated with the root code, transmitting, to a large language machine (LLM) engine, the at least portion of text from the document, the obtained child codes, and a prompt instructing the LLM engine to determine one or more specific ontology codes based on the at least portion of the text utilizing the obtained child codes, and receiving, from the LLM engine output that includes one or more specific ontology code.

### FIELD

The present disclosure relates generally to determining ontology codes utilizing a large language model engine.

### BACKGROUND

Electronic record keeping generally includes databases of documents that related to a specific context. Such context may be, for example, medical, legal, or financial (e.g., accounting, tax) contexts. In one such example of a medical context, medical record systems may include medicals records documents for particular patients that may include notes, such as in a “clinic visit note” often recorded after a family doctor visit, numerous diagnostic tests and procedures in medicine are similarly documented in free-text form, such as in radiology, pathology, and operative notes.

In some contexts, it may be useful to determine certain standardized, context-specific codes or classifications, to certain documents and/or entities associated with one or more documents. These standardized, context-specific codes are referred to generally as “ontology codes”in the present disclosure.

For example, in a medical context, the International Classification of Diseases version 10 Clinical Modification (ICD-10 /ICD-10 -CM), referred to herein as “ICD-10 ”, is the World Health Organization's (WHO) medical coding ontology which is the standard for used by health insurance companies for reimbursement of healthcare related expenses. In general, the more specific an ICD-10 code that can be generated from a clinical note, the ‘better’ the coding is, and the ‘better’ the reimbursement may be. Thus, people are highly incentivized to get the most accurate ICD-10 codes possible.

However, determining specific ontology codes accurately can be a time-consuming process that requires manual review by someone knowledgeable about the context in order to interpret meaning from the documents and knowledgeable about the specific ontology codes for that context in order to match the interpreted meaning with the applicable code(s).

In order to reduce the time and expense of determining codes related to documents in an electronic database, it is desirable to have computers systems that are configured to make such determinations. However, it is challenging to obtain accurate determinations using conventional computer systems for a number of reasons.

For example, context-specific documents rarely utilize language that matches the language utilized in the specific ontology codes, making it difficult to match in an intuitive manner the text included in the documents with the language included in ontology codes. In addition, documents stored in electronic document records may include often include unstructured text data, or “free-text”, that can be challenging for computer systems, including LLMs, to extract and interpret meaning from. For example, documents in a medical context often include free-text, such as “clinic visit notes” that are recorded after a family doctor visit, for example. In addition, numerous diagnostic tests and procedures in medical contexts may be similarly documented in free-text form, such as in radiology, pathology, and operative notes. such as a doctor's handwritten notes.

Improvements in computer-implemented processes for determining ontology coding are desired.

### SUMMARY

According to one aspect of an embodiment, the present disclosure provides a method that includes identifying, using context-specific natural language processor (NLP), an ontology code associated with at least a portion of text from a document, determining a root code associated with the identified ontology code, obtaining, from a database, child codes associated with the root code, transmitting, to a large language machine (LLM) engine, the at least portion of text from the document, the obtained child codes, and a prompt instructing the LLM engine to determine one or more specific ontology codes based on the at least portion of the text utilizing the obtained child codes, and receiving, from the LLM engine output that includes one or more specific ontology code.

In an example, the method further includes generating, utilizing a fine-tuned language model engine, structured data based on the at least portion of text from the document by identifying, in the at least portion of text, a span of text, identifying a concept that is associated with text included in the identified span of text, and wherein the structured data includes the identified concept and an identification of the identified span of text associated with the identified concept, wherein the structured data is transmitted to the LLM engine with the at least portion of text from the document, the obtained child codes, and the prompt, wherein the prompt instructs the LLM engine to determine the specific ontology code for the at least portion of the text utilizing the structured data.

In an example, obtaining child codes associated with the root code comprising performing a wildcard search for child codes that match the root code.

In an example, the prompt further instructs the LLM engine to generate reasons for each of the determined one or more specific ontology codes, and receiving the output from the LLM engine comprises receiving, for each of the determined one or more specific ontology codes, the generated reasons.

In an example, the document is a medical records document, and the context-specific NLP is specific to the medical field.

In an example, the identified and specific ontology codes comprise ontology codes set forth in the International Classification of Diseases version 10 Clinical Modification (ICD-10), and the database is a database that includes the ICD-10 codes.

According to a further aspect of an embodiment, the present disclosure provides an apparatus that includes at least one processor, at least one memory stored instructions wherein the instructions, when executed by the at least one processor, cause the processor to identify, using context-specific natural language processor (NLP), an ontology code associated with at least a portion of text from a document, determine a root code associated with the identified ontology code, obtain, from a database, child codes associated with the root code, transmit, to a large language machine (LLM) engine, the at least portion of text from the document, the obtained child codes, and a prompt instructing the LLM engine to determine one or more specific ontology codes based on the at least portion of the text utilizing the obtained child codes, and receive, from the LLM engine output that includes one or more specific ontology code.

In an example, the instructions, when executed by the at least one processor, further cause the processor to generate, utilizing a fine-tuned language model engine, structured data based on the at least portion of text from the document by identify, in the at least portion of text, a span of text, identify a concept that is associated with text included in the identified span of text, and wherein the structured data includes the identified concept and an identification of the identified span of text associated with the identified concept, wherein the structured data is transmitted to the LLM engine with the at least portion of text from the document, the obtained child codes, and the prompt, wherein the prompt instructs the LLM engine to determine the specific ontology code for the at least portion of the text utilizing the structured data.

In an example, the instructions that, when executed by the at least one processor, cause the processor to obtain child codes associated with the root code comprise instructions that, when executed by the at least one processor, cause the processor to perform a wildcard search for child codes that match the root code.

In an example, the prompt further instructs the LLM engine to generate reasons for each of the determined one or more specific ontology codes, and the instructions that, when executed by the at least one processor, cause the processor to receive the output from the LLM engine comprise instructions that, when executed by the at least one processor, cause the processor to receive, for each of the determined one or more specific ontology codes, the generated reasons.

In an example, the document is a medical records document, and the context-specific NLP is specific to the medical field.

In an example, the identified and specific ontology codes comprise ontology codes set forth in the International Classification of Diseases version 10 Clinical Modification (ICD-10), and the database is a database that includes the ICD-10 codes.

According to a further aspect of an embodiment, the present disclosure provides a computer readable medium having stored thereon computer-readable instructions that, when executed by at least one processor, cause the processor to identify, using context-specific natural language processor (NLP), an ontology code associated with at least a portion of text from a document, determine a root code associated with the identified ontology code, obtain, from a database, child codes associated with the root code, transmit, to a large language machine (LLM) engine, the at least portion of text from the document, the obtained child codes, and a prompt instructing the LLM engine to determine one or more specific ontology codes based on the at least portion of the text utilizing the obtained child codes, and receive, from the LLM engine output that includes one or more specific ontology code.

In an example, the instructions, when executed by the at least one processor, further cause the processor to generate, utilizing a fine-tuned language model engine, structured data based on the at least portion of text from the document by identify, in the at least portion of text, a span of text, identify a concept that is associated with text included in the identified span of text, and wherein the structured data includes the identified concept and an identification of the identified span of text associated with the identified concept, wherein the structured data is transmitted to the LLM engine with the at least portion of text from the document, the obtained child codes, and the prompt, wherein the prompt instructs the LLM engine to determine the specific ontology code for the at least portion of the text utilizing the structured data.

In an example, the instructions that, when executed by the at least one processor, cause the processor to obtain child codes associated with the root code comprise instructions that, when executed by the at least one processor, cause the processor to perform a wildcard search for child codes that match the root code.

In an example, the prompt further instructs the LLM engine to generate reasons for each of the determined one or more specific ontology codes, and the instructions that, when executed by the at least one processor, cause the processor to receive the output from the LLM engine comprise instructions that, when executed by the at least one processor, cause the processor to receive, for each of the determined one or more specific ontology codes, the generated reasons.

In an example, the document is a medical records document, and the context-specific NLP is specific to the medical field.

In an example, the identified and specific ontology codes comprise ontology codes set forth in the International Classification of Diseases version 10 Clinical Modification (ICD-10), and the database is a database that includes the ICD-10 codes.

The term “non-transitory,” as used herein, is a limitation of the medium itself (i.e., tangible, not a signal) as opposed to a limitation on data storage persistency (e.g., RAM vs. ROM).

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a schematic diagram showing a system in accordance with an aspect of an embodiment.

FIG. 2 is a flowchart showing a method in accordance with an example embodiment.

FIG. 3 is a schematic diagram showing components of one or more of the example embodiments.

### DETAILED DESCRIPTION

For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

Generally, the present disclosure provides a method and apparatus for providing a prompt to a large language model (LLM) engine, which may also be referred to herein simply as “LLM”, to determine ontology codes from at least a portion of text from a document.

The conventional approach for having an LLM engine perform any type of document review and/or analysis in a particular context, such as medical, legal, and financial contexts, primarily involves creating the LLM engine. In addition to standard training the LLM engine for generic generative purposes, creating the LLM engine may optionally include training the LLM engine specifically for the context for which it is intended to be used by. For example, a LLM engine may be trained specifically for use in a medical context utilizing medical documents or utilizing medical question-answer sets or other tasks to configure the LLM engine to specifically answer medical questions and/or perform tasks specific to the medical context.

Although the present disclosure describes example embodiments in terms of the medical context, this is for illustrative purposes only and it is understood that the same concepts described herein may be applicable to any other context that utilizes hierarchical ontology codes where linking mentions in text of a document to context-specific ontology codes may be desirable, which context may include, for example, legal, accounting, financial (e.g., tax), business, and so forth.

As noted previously, a challenge with having LLM engines identify specific ontology codes is that the text contained within a document rarely matches the language included in the description for each specific ontology code.

For example, the ICD-10 code for “Type 2 Diabetes” without other information is “E11 —Type 2 diabetes mellitus”. Identifying that generic code may be relatively straightforward. However, there are often a significant number of additional codes, referred to in the present disclosure as “child codes” below each “root code”. For example, E11.4 relates to Type 2 diabetes mellitus with neurological complications, which is a child code of E11—Type 2 diabetes mellitus. Further, code E11.4 may itself include addition child codes that stem from the E11.4 code, in which each child code includes specific types of neurological conditions, as follows:

However, no clinician would write that a patient has “Type 2 diabetes mellitus with diabetic neuropathy, unspecified” in a report, which makes computer-implemented identification of these ICD-10 codes utilizing medical record documents challenging. Conventionally, a human medical coder may be tasked with identifying in one sentence that the patient has Type 2 diabetes, and then in another sentence that the patient has diabetic neuropathy, and would then put these together into a single E11.40 code of “Type 2 diabetes mellitus with diabetic neuropathy, unspecified”. However, as noted previously, such human encoding requires specialized, context-specific knowledge and is extremely time consuming.

A further challenge with utilizing computers, such as LLM engines, to identify specific ontology codes is that the information within the documents that is relevant to specific ontology codes may be difficult for an LLM to interpret and/or make meaning of. For example, the relevant information may be in separate portions of texts, or otherwise disjointed. For instance, in the above example, the relevant information was provided in two separate sentences. Additionally, as described previously, the document may include unstructured data or “free-text” which may also be challenging for an LLM to interpret and/or make meaning of. Additionally, LLMs are known to be vulnerable to “hallucinations”, which refers to an LLM generate, or make up, factually incorrect information when performing a task, or to support the generated output. The task of determining ontology codes by an LLM may be particular susceptible to hallucinations because ontology codes have a very non-transparent connection between the number itself and the meaning assigned to that number. Additionally, ontology codes, such as ICD-10 codes in a medical context, may occur infrequently in context-specific text utilized for training LLMs when compared to other numbers, or numerical data, that occurs in this text, resulting in conventional LLMs generally lacking sufficient training with regard to ontology codes to determine these ontology codes accurately.

One conventional approach that may be utilized to attempt to determine ontology codes relevant to text of a document utilizing machine learning, such as LLMs, may be utilizing what is referred to as retrieval augmented generation (RAG). A RAG approach attempts to improve output of an LLM by using a language model to retrieve, based on the prompt that is provided to the LLM, passages of verifiable information from a collection of documents and/or a knowledge base of facts that are assumed to contain trusted information. The portions of those documents and/or knowledge base are retrieved using the language model by matching the content in those documents and/or knowledge base to the question or task included in the prompt. The language model used might be fine-tuned to the particular context of the collection of documents and/or the knowledge base. The portions from the documents and/or knowledge base that are retrieved by the language model are then sent to the LLM engine with the original prompt in order to provide context to the LLM such that the LLM performs the task associated with the prompt utilizing the retrieved portions of the documents and knowledge base. Conventional RAG approaches utilized for determining ontology codes may result in, for example, the entire set of ontology codes being provided together with the text of the document.

However, such approaches suffer from the same challenges as outlined above that the language included in the text of the document rarely matches language used in the description of each ontology code.

The present disclosure addresses at least some of these challenges by, first, utilizing a context-specific natural language processor (NLP) engine to identify an ontology code associated with the at least portion of the text. A root code is then determined for the identified ontology code, and child codes associated with the root codes are then obtained from a database of ontology codes. The at least portion of the text and only the obtained child codes are transmitted to an LLM with a prompt instructing the LLM to determine one or more specific ontology codes based on the at least portion of the text utilizing the obtained child codes.

By performing an initial determination of the ontology code using a context-specific NLP to obtain the potentially relevant child codes, and providing only these obtained child codes to the LLM, the LLM is able to focus the analysis it performs in relation to these specific child codes that are relevant to the document that is utilized for determining for determining the specific ontology code, which may result in a more accurate and/or more specific ontology determination than, for example, could be achieve by the context-specific NLP engine, or by an LLM that that not been provided with a list of only the relevant child codes obtained based on the initial ontology code determination of the context-specific NLP engine, to consider when making its determination.

In some examples, at least some of these above-noted challenges are address by further utilizing a fine-tuned language model to generate structured data based on the at least portion of the text, and transmitting the resulting structured data to the LLM with the obtained child codes and at least portion of the text. In such examples, the prompt further instructs the LLM to determine the one or more specific ontology codes utilizing the structured data, in addition to the obtained child codes and the at least portion of the text.

The structured data that is generated may, for example, identify concepts that are associated with spans of text that are included in the text of the document for which ontology codes are being determined. Alternatively, or additionally, the structured data may, for example, disambiguate ambiguous terms in the text of the document. Alternatively, or additionally, the structured data may identify multiple different concepts that are included in text of the document and identify a relationship between the different concepts.

In this way, the structured data is a reference that the LLM engine may utilize, similar to, for example, a legend or a key, when identifying specific ontology codes for the text in the document. By including the structured data, in addition to the obtained child codes, the LLM engine may better interpret and/or make meaning of the text in the document, which is informed by the structured data, to better overcome the challenges that language in the text of the document is unlikely to follow or match the language used in the ontology codes.

As noted previously, it may be desirable to determine the most specific ontology code that is applicable in a particular case, which most specific ontology codes being useful for any downstream applications of determined ontology code such as, for example, determining risk and/or reimbursements such as in, for example, a medical insurance application.

The children of a concept in the ontology are more specific concepts (and the parent is a more general concept). To determine the more specific concepts, i.e., more specific ontology codes, it may be desirable to consider knowledge from the entire document or portion of text from the document being coded. In particular, it may be desirable to know how multiple concepts included at different parts of the text relate to the correct identification of a child concept code. Providing the document text, plus all the child codes, plus, in some embodiments, the structured data related to the document text, enables providing the LLM with a prompt that asks the LLM to correlate these multiple sources of information and, based on the multiple sources of information, determine which is the correct child code to the provided text, which may result in more accurate determination of specific ontology codes compared to conventional approaches described previously.

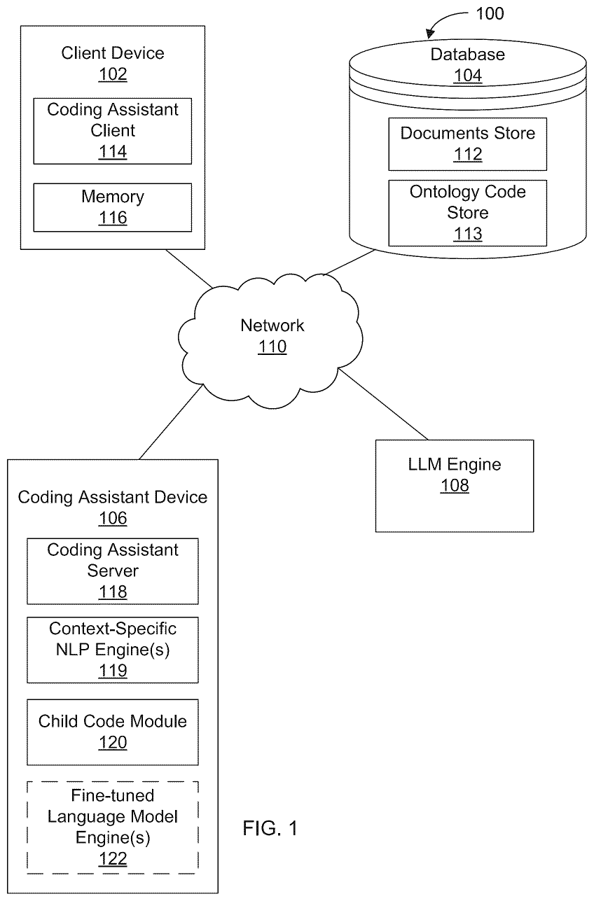

Referring now to FIG. 1, schematic representation of an example system 100 for instructing a LLM engine to identifier specific ontology codes is shown. The system 100 includes a client device 102, a database 104, a coding assistant device 106, and a LLM engine 108 that communicate with each other via a network 110. The network 110 may be any suitable wired or wireless network, or combination of wired and wireless networks including, for example, a local area network (LAN), or a wide area network (WAN), or a combination thereof.

The database 104 may include a document store 112 that stores electronic documents and an ontology code store 113 that stores ontology codes related to a specific context such as, for example, the ICD-10 utilized in medical contexts. The document store 112 may be, for example, part of an electronic record keeping system, or any other suitable document management system, that stores records for a particular context such as, for example, medical context, legal context, financial context, business context, and the like.

The client device 102 may include a coding assistant client 114 that may be utilized to generate requests to perform ontology coding on text in one or more documents. The coding assistant client 114 may include a graphical user interface that is displayed on a display (not shown) of the client device 102 to enable the user of the client device to input text, or otherwise make a selection within the user interface, to be initiate an ontology coding.

The coding assistant client 114 also enables one or more input documents to be included with the instructions to initiate an ontology coding. The input documents may be stored in a memory 116 of the client device, or in one or more remote databases, such as the document store 112 of the database 104. The coding assistant client 114 may retrieve copies of the input documents from the memory 116 or the database 104 and include the copies of the input documents with the instructions to initiate an ontology coding. Alternatively, the instructions transmitted by the coding assistant client 114 may include references or pointers to where the documents are stored.

The instructions may instruct the ontology coding to be performed on the entirety of the text of the one or more documents, or may instruct that the ontology coding be performed on a portion of the text contained in the one or more documents. For example, a user select a portion of text, and that only portion that portion of text, or an indication of the portion of text together with the one or more documents, may be transmitted by the coding assistant client 114. In an example, if one or more documents are included, or otherwise indicated, together with the instructions, without any particular indication of a portion of the text, then the ontology coding will be performed on the entirely of the contents of the one or more documents.

The coding assistant client 114 may be, for example, an application that is stored and executed at the client device 102, or may be a web-browser executed at the client device 102 to access a web-based application hosted on a server.

The coding assistant device 106 includes a coding assistant server 118, one or more context specific natural language processing (NLP) engine(s) 119, a child code module 120, and, in some embodiments, one or more a fine-tuned language model engine(s) 122.

The coding assistant server 118 is configured to interface with the coding assistant client 114 at the client device 102. The coding assistant server 118 may provide the graphical user interface that may be displayed at the client device 102 in some embodiments. The coding assistant server 118 may host the web-based application that is accessed when the coding assistant client 114 is implemented as a web browser that accesses the web-based application, as previously described. The coding assistant server 118 may receive instructions from the coding assistant client 114 to initiate a process of determining ontology codes. The coding assistant server 118 may provide selectable features to the coding assistant client 114 via, for example, a graphical user interface displayed on the coding assistant client 114, such that instructions are received at the coding assistant server 118 by selecting one of the selectable features. In an example, the coding assistant server 118 may receive text input by a user into the coding assistant client 114, determine that an ontology coding process is being to be initiated based on the text, and initiate the ontology coding process.

The coding assistant server 118 may receive the instructions from the coding assistant client 114 of the client device 102 together with one or more documents. As described previously, the coding assistant server 118 may receive copies of the one or more documents together with the instructions from the coding assistant client 114. Alternatively, or additionally, the coding assistant server 118 may receive indications of one or more documents, such as, for example, references or pointers to the one or more documents stored in the memory 116 or the database 104, in which case the coding assistant server 118 may retrieve copies of the one or more documents utilizing the references or pointers.

In other embodiments, a user may provide the instructions directly to the coding assistant device 106 utilizing a user input device (not shown) of the coding assistant device. For example, a display (not shown) of the coding assistant device may display a graphical user interface that the user may interact with, via a user input device, to input instructions to perform ontology coding and select one or more documents associated with the instructions.

The context-specific NLP engine 119 is configured receive the one or more documents, or one or more portions of text from the one or more documents, and identify an ontology code associated with at least a portion of text from the document. The context-specific NLP engine 119 may be configured to perform a process of “entity linking” in order to identify an ontology code. The context-specific NLP engine 119 may be configured to perform such entity linking through training using training datasets that include text as well as ontology codes related to that text. The context-specific NLP engine 119 may be configured to perform entity linking using a context window of a single sentence, such that the text in the one or more documents is analyzed sentence by sentence to attempt to identify a match with an ontology code.

In an example, the coding assistant device 106 may include context-specific NLP engines 119. For example, context-specific NLP engines 119 may be specific to different contexts such that one particular context-specific NLP engine 119 may be utilized to identify an initial ontology code in one context, and another context-specific NLP engine 119 may be utilized to identify an initial ontology code in another context. Alternatively, or additionally, two or more of the context-specific NLP engines 119 may each include different NLPs that are directed to the same context.

The child code module 120 is configured to receive the initial ontology code determined by the context-specific NLP engine 119, and determine a root code associated with the initial ontology code, then obtain child codes associated with the determined root code by, for example, performing a query of a database of ontology codes such as, for example, the ontology code store 113 of database 104, as described in more detail below. Alternatively, the ontology code store may be stored in a memory (not shown) of the coding assistant device 106.

Determining the “root code” may include determine the most generic code from which the various sub-codes, or child codes, stem. In an example of a medical context in which the ICD-10 provides the ontology codes, the initial ontology code determined by the context-specific NLP engine 119 is 37 E11.40—Type 2 diabetes mellitus with neurological complications, unspecified” as described previously. In this example, the root code may be determined to be E11.4, the child codes of which include ICD-10 codes for all neurological complications of type 2 diabetes. In another example, the root code may be determined by the child code module 120 to be 37 E11-Type 2 diabetes mellitus”, which is a much broader root code than E11.4 and the child codes of which include all of the ICD-10 codes for all Type 2 diabetes.

The child codes may be determined by the child code module 120 by performing a search of, for example, the ontology code store 113 of the database 104. For example, the child code module 120 may perform a wildcard search in which all of the characters other than the root character are replaced with a wildcard character, i.e., “*”. For example, if the determined root code is “E11”, then a search of the ontology code store 113 for ontology codes that match “E11*” may be performed, which would return all of the child codes associated with E11.

The coding assistant server 118 may optionally provide the one or more documents, or one or more portion(s) of text contained in the one or more document, to a fine-tuned language model engine 122 included at the coding assistant device 106. The fine-tuned language model 122 is configured to generate structured data associated with at least one of the one or more documents, or one or more portions of text contained in the one or more documents. As described in more detail below, the fine-tuned language model engine 122 may generate the structured data by linking spans of text found in the one or more documents, or one or more portions of text contained in the one or more documents, to context-specific concepts. In particular, spans of text may be disambiguated to specific concepts related to the context such as, for medical contexts, specific medical concepts.

The fine-tuned language model engine 122 may include one or more language models that are customized specific to a particular context that is associated with the one or more documents. For example, the fine-tuned language model engine 122 may include a pre-trained language model that is fine-tuned to perform context-specific language processing tasks such as, for example, entity linking to context-specific ontologies and relation finding for various context-specific relation finding tasks. This fine-tuning may include, for example, further training the language mode using expert human feedback such as, for example, human labelled data, and information from context-specific ontologies. For example, in a medical context, the fine-tuned language model engine 122 may be trained using data annotated by medical experts and linking them to concepts from a medical ontology such as, for example, SNOMED-CT.

In an example, the coding assistant device 106 may include multiple fine-tuned language model engines 122. For example, two or more of the fine-tuned language model engines 122 may be specific to different contexts such that one particular fine-tuned language model engine 122 may be utilized to generate structured data in one context, and another fine-tuned language model engine 122 may be utilized to generate structured data in another context. Alternatively, or additionally, two or more of the fine-tuned language model engines 122 may each include different language models that are directed to the same context.

The coding assistant server 118 may the transmit to the LLM engine 108 the one or more documents, or one or more portions of text contained in the one or more documents, the obtained child codes, and the structured data if structured data is generated based on the one or more documents, or one or more portions of text contained in the one or more documents the prompt, the one or more input documents, together with a prompt instructing the LLM engine 108 to determine one or more specific ontology codes based on the one or more documents, or one or more portions of text contained in the one or more documents, the obtained child codes, and, if included, the structured data.

The LLM engine 108 may be configured specific to the particular context related to the ontology codes being determined. For example, in a medical context, the LLM engine 108 may be specifically trained utilizing medical documents or utilizing medical question-answer sets or other tasks to configure the LLM engine 108 to specifically answer medical questions and/or perform tasks specific to the medical context, including determining medical ontology codes, such as ICD-10 codes.

LLM engines, such as LLM engine 108, are based on next-token prediction and, while they might have “knowledge” that certain concepts exist within the context, they do not disambiguate which concepts exist in a document until the LLM engine has to predict a token in the output. When included with the prompt, the structured data in the present disclosure provides metadata, in addition to the one or more documents, or one or more portions of text contained in the one or more documents, and the obtained child codes, that augments the prompt with context-specific ontology-based disambiguation concepts generated from the one or more documents, or one or more portions of text contained in the one or more documents. The structured data may be utilized by the LLM engine 108 as a reference, similar to, for example, a key or legend, when determining the specific ontology code(s) utilizing the one or more documents, or one or more portions of text contained in the one or more documents, and the obtained child codes. Use of the structured data, in addition to the one or more documents, or one or more portions of text contained in the one or more documents, and the obtained child codes, may result in more accurate determination of ontology codes and/or more specific ontology codes being determined compared to determinations without structured data by, for example, reducing the instances in which the LLM engine 108 is forced to attempt to disambiguate concepts related to the text of the one or more documents.

Although the concepts included in the structured data may be based on context-specific ontology, which ontology may be similar to a knowledge base that is used in the RAG approach that was previously described, the obtained child codes and, if provided, the structured data generated by the custom fine-tuned language models, such as the fine-tuned language model engine 122 described previously, is used to augment the one or more documents, or one or more portions of text contained in the one or more documents, with the relevant child codes and, if structured data is provided, context-specific concepts, to assist the LLM engine 108 to more accurately and/or more specifically determine ontology codes. This is in contrast to RAG approaches that merely provide generic information related to the context generally that is retrieved from the general knowledge base, such as source documents. The result of the present disclosure does not rely on retrieval of generic source documents, but rather provides information that is determined to be specifically related to the one or more documents, or one or more portions of text contained in the one or more documents, to assist in the LLM engine 108 to make a more accurate and/or more specific determination of ontology codes.

In an example, the LLM engine 108 may determine the specific child ontology code by utilizing structured data and the tree of child codes that provided with the prompt, as well as the location in the text that relates to the parent code, i.e., the portion of the text for which the initial ontology code was determined, then, utilizing the text of the entire content of the one or more documents, or the portion of text of the document, provided with the prompt, making a determination of the specific child code that is most appropriate for the entire content of the one or more documents, or the portion of the text of the one or more documents. This is in contrast to the identification of the initial ontology code which is determined by the context-specific NLP engine 119 analyzing individual sentences of the text.

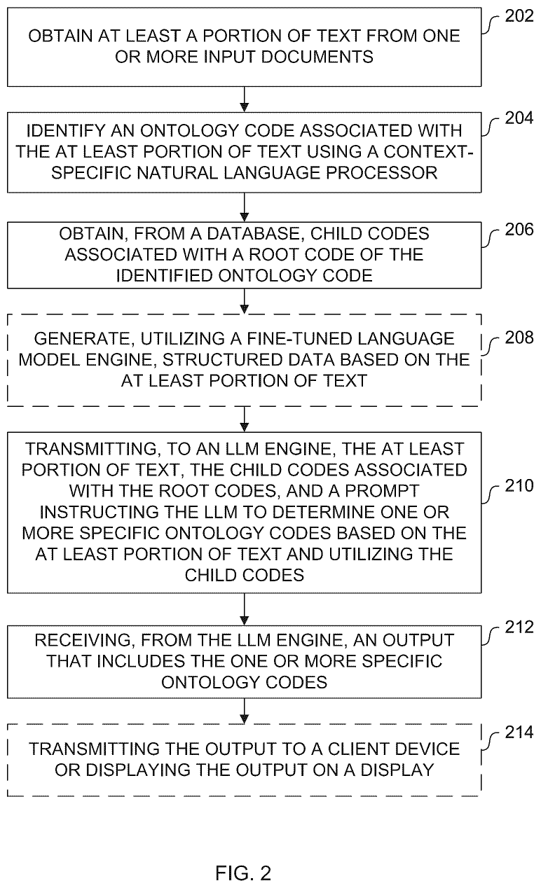

Referring now to FIG. 2, a flow chart showing an example method or process for instructing an LLM engine to determine specific ontology codes is shown. The example method or process may be performed by a coding assistant device such as, for example, the coding assistant device 106 described previously with reference to FIG. 1. The method or process may be performed by one or more processors of the coding assistant device that execute computer-readable code stored in a non-transitory memory of the coding assistant device, the computer-readable code providing instructions to the one or more processor for performing the method or process.

At 202, at least a portion of text from one or more documents is obtained. The portion of text from one or documents may be a portion of text received together with instructions received from a client device, such as client device 102 described previously with reference to FIG. 1 to perform ontology coding on the portion. The portion may be a part of the text included in the one or more documents, or may be all of the text included in the one or more documents. Alternatively, the instructions received from a client device may include a reference to the portion of text from the one or more document, such as a reference or a pointer to one or more documents stored in a document store of a database, such as the document store 112 of the database 112 described previously, or stored in a memory of the client device, such as the memory 116 of the client 102 described previously, and, possibly information indicating the text in the one or more documents for which coding is to be performed in the event that less than all of the text of the one or more documents is to be subject to the ontology coding. Alternatively, the instructions and the portion of the text, or the reference or point to the one or more documents and the information indicating the relevant text, may be received from an input device connected to the coding assistant client.

At 204, an ontology code associated with the at least portion of text is identified using a context-specific natural language processor (NLP). The context-specific natural processor may be the context-specific NLP engine 119 as previously described with reference to FIG. 1. As described previously, the context-specific NLP engine 119 may, for example, utilize an entity linking process that analyzes the at least portion of the text sentence by sentence to identify the ontology code at 204.

At 206, child codes associated with the ontology code identified at 204 are obtained from a database. As described previously, child codes may be determined by performing a query of an ontology code store for codes that include a root code associated with the identified ontology code. For example, a search in which a wild card character is added to the root code is performed of an ontology code store, such as the ontology code store 113 of the database 104 as described previously, or an ontology code store in another database such as, for example, in memory of the coding assistant device.

Optionally, at 208, structured data based on the at least portion of text is determined. As described previously, the structured data may be generated utilizing a fine-tuned language model. The fine-tuned language model may be the fine-tuned language model engine 122 described previously with reference to FIG. 1.

In an example, the fine-tuned language model engine may be specific to a context that is associated with the at least portion of the text. The fine-tuned language model may be trained using, for example, ontologies related to the context and/or other data that specific to the context. For example, the fine-tuned language model may be a pre-trained language model that is fine-tuned to perform context-specific language processing tasks such as, for example, entity linking to context-specific ontologies and relation finding for various context-specific relation finding tasks. This fine-tuning may include, for example, further training the language mode using expert human feedback such as, for example, human labelled data, and information from context-specific ontologies.

For example, in a medical context, the fine-tuned language model may be trained using data annotated by medical experts and linking them to concepts from a medical ontology such as, for example, SNOMED-CT.

The structured data may identify concepts that are associated with spans of text that are present in at least portion of text, may disambiguate ambiguous terms in the at least portion of text, may identify multiple concepts that are included in the at least portion of text and identify a relationship between the concepts.

Generating the structured data at 208 may include identifying spans of text in the at least portion of text and linking the identified spans of text to one or more context-specific concepts.

In an example, spans of text that include ambiguous terms may be identified, and the ambiguous terms may be disambiguated to specific concepts related to the context such as, for example, specific medical concepts in a medical context. In some examples, two or more concepts may be associated with an identified span of text, or spans of texts, and the structured data may include, in addition to the two more concepts associated with the span of text, a relationship among the two or more concepts.

Generating structured data may include identifying the text spans related to a concept in the one or more input documents, performing disambiguation for ambiguous strings in the text span, and including the structured data information linking ambiguous strings to the correct concept in the ontology. In a medical context, for example, the generated structured data may also include medical entities for open-ended information like polarity (negation), measurements, temporality, qualifiers and identifying the person/people who experiences the medical entity so that, for example, family history is not conflated by the LLM engine with patient history.

In an example, the structured data may be formatted as an array or a table in which each row may include the following: (1) a span of text from the at least portion of text, (2) a concept identifier from a context-specific ontology that relates to the span of text, and (3) the fully specified name and/or a short unambiguous description of the concept. In this way, the structured data disambiguates the spans of text in the at least portion of text with the appropriate concept based on the contextual information in the input document. In some examples, the structured data may include an additional array or table that that associates two or more concepts in a relationship.

In some examples, generating the structured data at 210 may comprise determining a context associated with the at least portion of text, then determining a fine-tuned language model engine that is specifically configured for that context from among a plurality of fined-tuned language model engines that are each specific to a particular context, as described previously with reference to the fine-tuned language model engine(s) 122. In this case, multiple fine-tuned language model engines may be provided in, or in association with, the coding assistant device that are specific to different context. Then depending on the particular context, the received one or more input documents and received prompt, a particular one of the fine-tuned language model engines may be utilized to generate the structured data. In this way, a single coding assistant device may be utilized to receive portions of text that relate to different contexts, generate context-specific structured data.

At 210, the at least portion of the text obtained at 202 and the child codes obtained by 206 are transmitted to an LLM engine, such as the LLM engine 108 described previously with reference to FIG. 1, together with a prompt instructing the LLM engine to determine one or more specific ontology codes utilizing the at least portion of the text, the child codes.

In examples in which structured data is generated at 208, the structured data is transmitted to the LLM engine at 210 together with the prompt instructing the LLM engine to determine the one or more specific ontology codes utilizing the structured data. The matched ontology concepts included in the structured data generated at 208 adds additional structured information and additional instructions to the LLM engine beyond the information included in the at least portion of text. This structured information may be used by the LLM engine to copy verifiable information from useful text spans in the at least portion of text, including for example, the ontology concept identifiers.

By causing the LLM engine to perform the determination of one or more specific ontology codes based on the at least one portion of text utilizing the structured data, the LLM engine may utilize the structured data as a reference to, for example, disambiguate ambiguous terms found in the at least portion of text. In this way, ambiguous terms and concepts found in the one or more input documents are disambiguated by a fine-tuned language model engine that is context-specific and trained to disambiguate concepts using context-specific ontologies, rather than the LLM engine being forced to perform such disambiguation, a task that it is not specifically trained to perform, i.e., disambiguate ambiguous information.

By including structured data together with the at least portion of text, as described above, the LLM engine may be better able to correlate and match the instruction in the prompt to the information included in the input documents and the structured information in the structured data order to follow the instructions included in the prompt to perform the determination of one or more specific ontology codes. The decision making in an LLM engine is a very complex process that may involve billions of parameters. The structured data that is provided to the LLM engine in accordance with embodiments of the present disclosure augment the input context of the LLM engine to better enable the next token prediction capabilities of a LLM engine to find the one or more specific ontology codes of the child codes in a vast solution space.

In some examples, the prompt may further instruct the LLM engine to provide reasons for the one or more specific ontology codes that it determines based on the at least portion of the text, the child codes, and, if provided, the structured data. The reasons for the LLM engine's decision may be helpful for transparency, which may be helpful for a user to determine how the LLM engine arrived at its determination and how reliable the determination is. Further, the transparency in the reasons may be utilized to, for example, fine the tune the LLM engine by, for example, identifying and adjusting assumptions made by the LLM engine, to provide more accurate determinations of specific ontology codes in instances where the LLM engine's determination was incorrect.

Causing the LLM engine to provide reasons for its determination of one or more specific ontology codes may result in more accurate determinations. For example, by providing reasons, the VLM engine is forced to better understand the context and content of the at least portion of the text, providing enhanced contextual understanding. This deeper understanding may help the LLM engine more accurately determine the one or more specific ontology codes. Additionally, when the LLM engine is forced to articulate its reasoning, it has to verify each step of its logic, which may provide error checking and correction that the LLM engine might not otherwise perform if it is not generating reasons for its determination. This process may cause the LLM engine to catch and correct errors that might have gone unnoticed if the LLM engine were simply providing the determination without explanation. Additionally, the LLM engine being required to provide reasons for its decisions may impose a structured approach to the task of determining one or more specific ontology codes, resulting in structured thinking which may cause the LLM engine considers all relevant factors systematically, leading to more accurate and reliable outputs.

Instructing the LLM engine to provide reasoning may enhance the accuracy of the determinations of one or more specific ontology codes by promoting a deeper and more structured engagement with the at least portion of the text, the child codes, and if provided the structured data, facilitating error detection and correction, and reinforcing logical decision-making processes. This methodical approach by the LLM engine may lead to a more precise identification and determination of one or more specific ontology codes for the at least portion of the text.

At 212, output is received from the LLM engine that includes the determine one or more specific ontology codes determined by the LLM engine in response to the prompt transmitted at 210. If the prompt transmitted to the LLM engine at 210 instructs the LLM engine to generate reasons for the determination of the one or more specific ontology codes, then the output received at 212 includes the reasons generated by the LLM engine.

Optionally, at 214, when the output is received at 212, the output may be transmitted to a client device, or displayed on a display. For example, the output may be transmitted to the client device that the original instructions to perform ontology coding was received from. The output may be transmitted by, for example, a coding assistant server to a coding assistant client at the client device. Alternatively, or additionally, the output may be displayed on a display. For example, if the original instructions to perform ontology coding were received from, for example, from a user via a user interface at the coding assistant device, then the output may be displayed to the user on a display of the coding assistant device. In other examples, the output of the LLM engine may optionally be transmitted to any other device including, for example, a database, such as database 104 described previously, for storage.

In an example, more than initial ontology codes may be determined at 204. In this case, steps 206, 210, and 212 may be iteratively performed for each of the initial ontology code determined at 204 such that the child codes for a first initial ontology code are determined at 206, and are transmitted to the LLM engine with a prompt, the at least portion of the text and, optionally, structured data at 210, and only after receiving the output at 212 is this process repeated for a second initial ontology code. Alternatively, all of the initial ontology codes identified at 204 may be processed together, such that child codes are all of the initial ontology codes are determined at 206, and all of these child codes are transmitted together to the LLM engine at 210 with the prompt, the at least portion of the text, and, optionally, the structured data. Alternatively, the process may be performed in parallel for each of the initial ontology codes determined at 204 such that, for example, the child codes of each of the identified initial ontology codes are transmitted to respective LLM engines at 210 together with a prompt, the at least portion of the text and, optionally, the structured data such that specific ontology codes for each of the identified initial ontology codes are determined in parallel.

The following describes a particular example of performing an ontology coding process on a portion of text from a document in a medical context utilizing, for example, a system similar to the system described previously with reference to FIG. 1, and a process similar to the process described previously with reference to FIG. 2.

In an example, the following portion of text from medical document is transmitted from a coding client device, such as coding client device 102, to a coding assistant device, such as coding assistant device 106:

### History Of Present Illness

The patient is a 61-year-old male with a history of diabetes type 2×5 years complicated by peripheral neuropathy, hyperlipidemia, and GERD, who presented to the emergency department on March 22nd with a chief complaint of epigastric pain and burning. The patient had exerted himself and had felt fatigued and lightheaded. At that point in time, he was also short of breath and slightly diaphoretic. He had an elevation of his ST elevations and old Q waves inferiorly on his EKG at that point in time. The patient was then cardiac catheterized, at which time he had a cardiac output of 5.15 with an index of 2.26. Assessment was severe LMCA three-vessel coronary artery disease with recent thrombotic occlusion distal RCA, severe left ventricular diastolic heart failure, and he had a balloon pump inserted. It was decided that the patient could undergo CABG by Dr. Sarkar. He underwent a CABG×4, LIMA to LAD with a jump graft, ramus, and he was diagnosed with a PDA. He had EF preoperatively of 30 percent.

### Past Medical History

His past medical history included diabetes with peripheral neuropathy, hyperlipidemia, coronary artery disease, and GERD.

An initial ontology code is determined for the example portion of text shown above using a context-specific NLP engine, such as context-specific MPL engine, that is trained specifically to a medical context. In the present example, the text string ‘diabetes type 2′ in the first sentence is identified as being the ICD-10 code of E11.9-TYPE 2 DIABETES MELLITUS WITHOUT COMPLICATIONS.

The identified initial ontology code “E11.9” is truncated to root code “E11”, and a query of an ontology code store, such as ontology code store 113, for all child codes under “E11” using, for example a wildcard search for all codes having “E11*”, where “*” is a wildcard character, the results of which query include the following example child codes:

E11.00-Type 2 diabetes mellitus with hyperosmolarity without nonketotic hyperglycemic-hyperosmolar coma (NKHHC)

E11.01-Type 2 diabetes mellitus with hyperosmolarity with coma

E11.10-Type 2 diabetes mellitus with ketoacidosis without coma

E11.11-Type 2 diabetes mellitus with ketoacidosis with coma

E11.21-Type 2 diabetes mellitus with diabetic nephropathy

E11.22-Type 2 diabetes mellitus with diabetic chronic kidney disease

E11.29-Type 2 diabetes mellitus with other diabetic kidney complication

E11.311-Type 2 diabetes mellitus with unspecified diabetic retinopathy with macular edema

E11.319-Type 2 diabetes mellitus with unspecified diabetic retinopathy without macular edema

E11.3211-Type 2 diabetes mellitus with mild nonproliferative diabetic retinopathy with macular edema, right eye

E11.3212-Type 2 diabetes mellitus with mild nonproliferative diabetic retinopathy with macular edema, left eye

E11.3213-Type 2 diabetes mellitus with mild nonproliferative diabetic retinopathy with macular edema, bilateral

E11.3219-Type 2 diabetes mellitus with mild nonproliferative diabetic retinopathy with macular edema, unspecified eye

E11.3291-Type 2 diabetes mellitus with mild nonproliferative diabetic retinopathy without macular edema, right eye

E11.3292-Type 2 diabetes mellitus with mild nonproliferative diabetic retinopathy without macular edema, left eye

E11.3293-Type 2 diabetes mellitus with mild nonproliferative diabetic retinopathy without macular edema, bilateral

E11.3299-Type 2 diabetes mellitus with mild nonproliferative diabetic retinopathy without macular edema, unspecified eye

E11.3311-Type 2 diabetes mellitus with moderate nonproliferative diabetic retinopathy with macular edema, right eye

E11.3312-Type 2 diabetes mellitus with moderate nonproliferative diabetic retinopathy with macular edema, left eye

E11.3313-Type 2 diabetes mellitus with moderate nonproliferative diabetic retinopathy with macular edema, bilateral

E11.3319-Type 2 diabetes mellitus with moderate nonproliferative diabetic retinopathy with macular edema, unspecified eye

E11.3391-Type 2 diabetes mellitus with moderate nonproliferative diabetic retinopathy without macular edema, right eye

E11.3392-Type 2 diabetes mellitus with moderate nonproliferative diabetic retinopathy without macular edema, left eye

E11.3393-Type 2 diabetes mellitus with moderate nonproliferative diabetic retinopathy

without macular edema, bilateral

E11.3399-Type 2 diabetes mellitus with moderate nonproliferative diabetic retinopathy

without macular edema, unspecified eye

E11.3411-Type 2 diabetes mellitus with severe nonproliferative diabetic retinopathy with

macular edema, right eye

E11.3412-Type 2 diabetes mellitus with severe nonproliferative diabetic retinopathy with

macular edema, left eye

E11.3413-Type 2 diabetes mellitus with severe nonproliferative diabetic retinopathy with

macular edema, bilateral

E11.3419-Type 2 diabetes mellitus with severe nonproliferative diabetic retinopathy with macular edema, unspecified eye

E11.3491-Type 2 diabetes mellitus with severe nonproliferative diabetic retinopathy

without macular edema, right eye

E11.3492-Type 2 diabetes mellitus with severe nonproliferative diabetic retinopathy

without macular edema, left eye

E11.3493-Type 2 diabetes mellitus with severe nonproliferative diabetic retinopathy

without macular edema, bilateral

E11.3499-Type 2 diabetes mellitus with severe nonproliferative diabetic retinopathy

without macular edema, unspecified eye

E11.3511-Type 2 diabetes mellitus with proliferative diabetic retinopathy with macular edema, right eye

E11.3512-Type 2 diabetes mellitus with proliferative diabetic retinopathy with macular edema, left eye

E11.3513-Type 2 diabetes mellitus with proliferative diabetic retinopathy with macular edema, bilateral

E11.3519-Type 2 diabetes mellitus with proliferative diabetic retinopathy with macular edema, unspecified eye

E11.3521-Type 2 diabetes mellitus with proliferative diabetic retinopathy with traction retinal detachment involving the macula, right eye

E11.3522-Type 2 diabetes mellitus with proliferative diabetic retinopathy with traction retinal detachment involving the macula, left eye

E11.3523-Type 2 diabetes mellitus with proliferative diabetic retinopathy with traction retinal detachment involving the macula, bilateral

E11.3529-Type 2 diabetes mellitus with proliferative diabetic retinopathy with traction retinal detachment involving the macula, unspecified eye

E11.3531-Type 2 diabetes mellitus with proliferative diabetic retinopathy with traction retinal detachment not involving the macula, right eye

E11.3532-Type 2 diabetes mellitus with proliferative diabetic retinopathy with traction retinal detachment not involving the macula, left eye

E11.3533-Type 2 diabetes mellitus with proliferative diabetic retinopathy with traction retinal detachment not involving the macula, bilateral

E11.3539-Type 2 diabetes mellitus with proliferative diabetic retinopathy with traction retinal detachment not involving the macula, unspecified eye

E11.3541-Type 2 diabetes mellitus with proliferative diabetic retinopathy with combined traction retinal detachment and rhegmatogenous retinal detachment, right eye

E11.3542-Type 2 diabetes mellitus with proliferative diabetic retinopathy with combined traction retinal detachment and rhegmatogenous retinal detachment, left eye

E11.3543-Type 2 diabetes mellitus with proliferative diabetic retinopathy with combined traction retinal detachment and rhegmatogenous retinal detachment, bilateral

E11.3549-Type 2 diabetes mellitus with proliferative diabetic retinopathy with combined traction retinal detachment and rhegmatogenous retinal detachment, unspecified eye

E11.3551-Type 2 diabetes mellitus with stable proliferative diabetic retinopathy, right eye

E11.3552-Type 2 diabetes mellitus with stable proliferative diabetic retinopathy, left eye

E11.3553-Type 2 diabetes mellitus with stable proliferative diabetic retinopathy, bilateral

E11.3559-Type 2 diabetes mellitus with stable proliferative diabetic retinopathy, unspecified eye

E11.3591-Type 2 diabetes mellitus with proliferative diabetic retinopathy without macular edema, right eye

E11.3592-Type 2 diabetes mellitus with proliferative diabetic retinopathy without macular edema, left eye

E11.3593-Type 2 diabetes mellitus with proliferative diabetic retinopathy without macular edema, bilateral

E11.3599-Type 2 diabetes mellitus with proliferative diabetic retinopathy without macular edema, unspecified eye

E11.36-Type 2 diabetes mellitus with diabetic cataract

E11.37X1-Type 2 diabetes mellitus with diabetic macular edema, resolved following treatment, right eye

E11.37X2-Type 2 diabetes mellitus with diabetic macular edema, resolved following treatment, left eye

E11.37X3-Type 2 diabetes mellitus with diabetic macular edema, resolved following treatment, bilateral

E11.37X9-Type 2 diabetes mellitus with diabetic macular edema, resolved following treatment, unspecified eye

E11.39-Type 2 diabetes mellitus with other diabetic ophthalmic complication

E11.40-Type 2 diabetes mellitus with diabetic neuropathy, unspecified

E11.41-Type 2 diabetes mellitus with diabetic mononeuropathy

E11.42-Type 2 diabetes mellitus with diabetic polyneuropathy

E11.43-Type 2 diabetes mellitus with diabetic autonomic (poly)neuropathy

E11.44-Type 2 diabetes mellitus with diabetic amyotrophy

E11.49-Type 2 diabetes mellitus with other diabetic neurological complication

E11.51-Type 2 diabetes mellitus with diabetic peripheral angiopathy without gangrene

E11.52-Type 2 diabetes mellitus with diabetic peripheral angiopathy with gangrene

E11.59-Type 2 diabetes mellitus with other circulatory complications

E11.610-Type 2 diabetes mellitus with diabetic neuropathic arthropathy

E11.618-Type 2 diabetes mellitus with other diabetic arthropathy

E11.620-Type 2 diabetes mellitus with diabetic dermatitis

E11.621-Type 2 diabetes mellitus with foot ulcer

E11.622-Type 2 diabetes mellitus with other skin ulcer

E11.628-Type 2 diabetes mellitus with other skin complications

E11.630-Type 2 diabetes mellitus with periodontal disease

E11.638-Type 2 diabetes mellitus with other oral complications

E11.641-Type 2 diabetes mellitus with hypoglycemia with coma

E11.649-Type 2 diabetes mellitus with hypoglycemia without coma

E11.65-Type 2 diabetes mellitus with hyperglycemia

E11.69-Type 2 diabetes mellitus with other specified complication

E11.8-Type 2 diabetes mellitus with unspecified complications

E11.9-Type 2 diabetes mellitus without complications

In total, under the current ICD-10 there are 86 child codes for the root code “E11”, which include the above codes.

Optionally, structured data is generated for the portion of the text by a fine-tuned language model engine. The structured data may be generated by instructing, for example utilizing a prompt, to generate structured data for the at least portion of the text. The instructions may be provided to the fine-tuned language model engine together with the at least portion of the text. The structured data may be in the format of the particular span of text from the portion of text, “text”, a concept identifier, “concept_id”, which relates to a particular concept in the ontology related to the context, in this example a medical context, and a description of the particular concept that is identified by the concept identifier, “description”.

A prompt is generated by the coding assistant device that includes instructions for the LLM engine to determine one or more specific ontology codes for the at least portion of the text. The prompt is then transmitted to the LLM engine together with the above portion of text, the above obtained child codes, and if optionally generated, the structured data associated with the portion of text.

In this example, the following example output is received from the LLM engine:
Based on the provided medical document and NLP output, here are the most specific ICD-10 codes I can determine for this patient's diabetes and related complications, with justifications:
E11.42 Type 2 diabetes mellitus with diabetic polyneuropathy. Justification: The patient has a history of “diabetes type 2 (SNOMED-ID: 44054006) complicated by peripheral neuropathy (SNOMED-ID: 302226006)”. Diabetic peripheral neuropathy is a type of diabetic polyneuropathy.
E11.59 Type 2 diabetes mellitus with other circulatory complications. Justification: The patient was found to have “severe LMCA three-vessel coronary artery disease (SNOMED-ID: 233817007)” and underwent CABG surgery. Coronary artery disease is a circulatory complication of diabetes.
E11.65 Type 2 diabetes mellitus with hyperglycemia. Justification: Although not explicitly stated, the patient likely had hyperglycemia, since this is common in uncontrolled diabetes and can contribute to complications like coronary artery disease (SNOMED-ID: 53741008).

As shown in this example the code of “E11.9-Type 2 Diabetes Mellitus Without Complications” determined by the context-specific NLP engine is not only suboptimal but is incorrect as this patient has neurologic and likely cardiovascular complications of diabetes. In the present example, the system and method described herein results in the identification of three relevant ICD-10 codes for the patient-E11.42, E11.59, and E11.65, and provides a reasonable justification for all of them, based on the portion of the text.

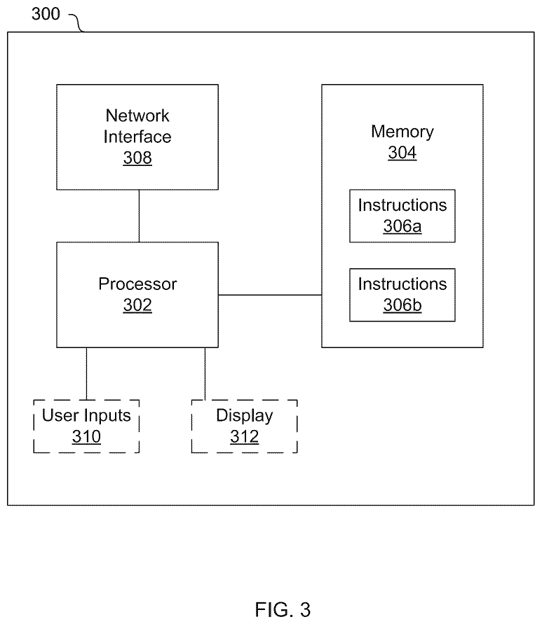

Referring to FIG. 3, a schematic diagram illustrating various physical and logical components of an exemplary apparatus 300 for a coding assistant device in accordance with an embodiment is shown. Although an example embodiment of the apparatus 300 is shown and discussed below, other embodiments may be used to implement examples disclosed herein, which may include components different from those shown. Although FIG. 3 shows a single instance of each component of the apparatus 300, there may be multiple instances of each component shown.

The apparatus 300 includes one or more processors 302, such as a central processing unit (CPU), a microprocessor, an application-specific integrated circuit (ASIC), a field-programmable gate array (FPGA), a dedicated logic circuitry, a graphics processing unit (GPU), a tensor processing unit, a neural processing unit, a dedicated artificial intelligence processing unit, a hardware accelerator, or any other suitable hardware processing circuitry, or combinations thereof. The one or more processors 302 may collectively be referred to as a processor 302.

The apparatus 300 also includes one or more memories 304 (collectively referred to as “memory 304″), which may include a volatile or non-volatile memory (e.g., a flash memory, a random-access memory (RAM), and/or a read-only memory (ROM)). The non-transitory memory 304 may store instructions for execution by the processor 302. In some embodiments, instructions 306 of a coding assistant device, a coding assistant server, one or more context-specific NLP engines, a child code module, and one or more fine-tuned language model engines, as described herein, such as the coding assistant device 106, the coding assistant server 118, the one or more context-specific NLP engines 119, the child code module 120, and the one or more fine-tuned language model engines 122, of the example system 100, may be stored in the memory 304, and the instructions 306 may be executed by the processor 302 to perform the actions or operations of the methods or processes described herein.

The apparatus 300 may also include one or more network interfaces 308 for connecting to a network, such as the network 110, for communication with, for example, a client device, such as client device 102 of the example system 100, a database, such as the database 104 of the example system 100, and a LLM engine, such as the LLM engine 108 of the example system 100.

The apparatus may optionally include a user input 310 for receiving input from a user of the apparatus 100 and a display 312. The user input 310 may be utilized, for example, for a user to interact with a graphical user interface displayed on the display 312 in order to input a instructions and/or select input documents or select at least a portion of text from an input document for which ontology coding performed by an LLM engine is desired.

In this case, the instructions may be received directly from the user, via the user input 310, rather than from a client device.

In some examples, the apparatus 300 may also include one or more electronic storage units (not shown), such as a solid state drive, a hard disk drive, a magnetic disk drive and/or an optical disk drive. In some examples, one or more datasets and/or modules may be provided by an external memory (e.g., an external drive in wired or wireless communication with the computing system 300) or may be provided by a transitory or non-transitory computer-readable medium. Examples of non-transitory computer readable media include a RAM, a ROM, an erasable programmable ROM (EPROM), an electrically erasable programmable ROM (EEPROM), a flash memory, a CD-ROM, or other portable memory storage. The storage units and/or external memory may be used in conjunction with memory 604 to implement data storage, retrieval, and caching functions of the apparatus 300.

The components of the apparatus 300 may communicate with each other via a bus. In some embodiments, the apparatus 300 may be a processing system implementing functionality of the coding assistant device described herein, such as the coding assistant device 106 of the example system 100 previously described with reference to FIG. 1. In some embodiments, the apparatus 300 may be distributed computing system and may include multiple computing devices in communication with each other over a network, as well as optionally one or more additional components. The various operations described herein may be performed by different computing devices of a distributed computing system in some embodiments. In some embodiments, the apparatus 300 may be a cloud computing system or may be a virtual machine provided by a cloud computing system.

Embodiments of the present disclosure enable determining one or more specific ontology codes based on at least a portion of text from a document. The determination of one or more specific ontology codes is performed by first determining an initial ontology code based on the portion of text by a context-specific NLP engine, then obtaining child codes associated with a root code of the initial ontology code. The portion of the text and the obtained child codes are sent to an LLM engine together with a prompt instructing the LLM engine to determine one or more specific ontology codes based on the at least portion of the text utilizing the obtained child codes.

The process may further include the generation of structured data by a fine-tuned language model based on the portion of text. The structured data may identify concepts that are associated with spans of text that are present in the portion of text, may disambiguate ambiguous terms in the portion of text, may identify multiple concepts that are included in the portion of text and identify a relationship between the concepts. The structured data is then provided to the LLM engine with the prompt, the portion of text and the child codes with instructions to cause the LLM engine to utilizing the structured data when determining the one or more specific ontology codes. Utilizing the structured data, generated by a fine-tuned language model, by the LLM engine while performing the task based on the one or more input documents may result in more accurate determination of the one or more specific ontology codes.

In other examples, the prompt instructs the LLM engine to provide reasons for the determination of the one or more specific ontology codes, which may further increase the accuracy of the determined one or more specific ontology codes by, for example, causing the LLM engine to perform error correction.

Embodiments of the present disclosure provide a technical solution to the technical problem of determining more accurate ontology codes for a portion of text by an LLM engine. By determining an initial ontology code, then determining child codes associated with the determined initial ontology code, then providing both the portion of text and the child codes to the LLM engine, more accurate determinations of ontology codes may be performed by the LLM engine, resulting in an improvement in the functioning of the overall computer system utilized to determine ontology codes. Further, having a fine-tuned, context-specific, language model engine that generates structured data utilizing the one or more input documents may enable the determination of ontology codes by a generalized LLM engine, i.e., not fine-tuned.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

As used in the present disclosure, the term “circuitry” may refer to one or more or all of the following: (a) hardware-only circuit implementations (such as implementations in only analog and/or digital circuitry) and (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions) and (iii) hardware circuit(s) and or processor(s), such as a microprocessor(s) or a portion of a microprocessor(s), that requires software (e.g., firmware) for operation, but the software may not be present when it is not needed for operation. This definition of circuitry applies to all uses of this term in this application, including in any claims. As a further example, as used in the present disclosure, the term circuitry also covers an implementation of merely a hardware circuit or processor (or multiple processors) or portion of a hardware circuit or processor and its (or their) accompanying software and/or firmware.

The functions, processes, and operations described herein may be performed in a different order, or may be performed concurrently with each other, or a combination thereof. Furthermore, one or more of the functions, processes, and operations may be optional or may be combined. It will be appreciated that the flow diagram shown in FIG. 2 and the various embodiments described with reference to FIG. 2, are examples only. Various operations and processes depicted therein may be omitted, may be reordered, may be combined, or a combination of reordered and combined.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A method comprising:
identifying, using context-specific natural language processor (NLP), an ontology code associated with at least a portion of text from a document;
determining a root code associated with the identified ontology code;
obtaining, from a database, child codes associated with the root code;
transmitting, to a large language machine (LLM) engine, the at least portion of text from the document, the obtained child codes, and a prompt instructing the LLM engine to determine one or more specific ontology codes based on the at least portion of the text utilizing the obtained child codes; and
receiving, from the LLM engine output that includes one or more specific ontology code.

2. The method according to claim 1, wherein the method further comprises:
generating, utilizing a fine-tuned language model engine, structured data based on the at least portion of text from the document by:
identifying, in the at least portion of text, a span of text;
identifying a concept that is associated with text included in the identified span of text; and
wherein the structured data includes the identified concept and an identification of the identified span of text associated with the identified concept;
wherein the structured data is transmitted to the LLM engine with the at least portion of text from the document, the obtained child codes, and the prompt, wherein the prompt instructs the LLM engine to determine the specific ontology code for the at least portion of the text utilizing the structured data.

3. The method of claim 1, wherein obtaining child codes associated with the root code comprising performing a wildcard search for child codes that match the root code.

4. The method of claim 1, wherein:
the prompt further instructs the LLM engine to generate reasons for each of the determined one or more specific ontology codes; and
receiving the output from the LLM engine comprises receiving, for each of the determined one or more specific ontology codes, the generated reasons.

5. The method of claim 1, wherein the document is a medical records document, and the context-specific NLP is specific to the medical field.

6. The method of claim 5, wherein the identified and specific ontology codes comprise ontology codes set forth in the International Classification of Diseases version 10 Clinical Modification (ICD-10 ), and the database is a database that includes the ICD-10 codes.

7. An apparatus comprising:
at least one processor;
at least one memory stored instructions wherein the instructions, when executed by the at least one processor, cause the processor to:
identify, using context-specific natural language processor (NLP), an ontology code associated with at least a portion of text from a document;
determine a root code associated with the identified ontology code;
obtain, from a database, child codes associated with the root code;
transmit, to a large language machine (LLM) engine, the at least portion of text from the document, the obtained child codes, and a prompt instructing the LLM engine to determine one or more specific ontology codes based on the at least portion of the text utilizing the obtained child codes; and
receive, from the LLM engine output that includes one or more specific ontology code.

8. The apparatus according to claim 7, wherein the instructions, when executed by the at least one processor, further cause the processor to:
generate, utilizing a fine-tuned language model engine, structured data based on the at least portion of text from the document by:
identify, in the at least portion of text, a span of text;
identify a concept that is associated with text included in the identified span of text; and
wherein the structured data includes the identified concept and an identification of the identified span of text associated with the identified concept;
wherein the structured data is transmitted to the LLM engine with the at least portion of text from the document, the obtained child codes, and the prompt, wherein the prompt instructs the LLM engine to determine the specific ontology code for the at least portion of the text utilizing the structured data.

9. The apparatus of claim 7, wherein the instructions that, when executed by the at least one processor, cause the processor to obtain child codes associated with the root code comprise instructions that, when executed by the at least one processor, cause the processor to perform a wildcard search for child codes that match the root code.

10. The apparatus of claim 7, wherein:
the prompt further instructs the LLM engine to generate reasons for each of the determined one or more specific ontology codes; and
the instructions that, when executed by the at least one processor, cause the processor to receive the output from the LLM engine comprise instructions that, when executed by the at least one processor, cause the processor to receive, for each of the determined one or more specific ontology codes, the generated reasons.

11. The apparatus of claim 7, wherein the document is a medical records document, and the context-specific NLP is specific to the medical field.

12. The apparatus of claim 11, wherein the identified and specific ontology codes comprise ontology codes set forth in the International Classification of Diseases version 10 Clinical Modification (ICD-10), and the database is a database that includes the ICD-10 codes.

13. A computer readable medium having stored thereon computer-readable instructions that, when executed by at least one processor, cause the processor to:
identify, using context-specific natural language processor (NLP), an ontology code associated with at least a portion of text from a document;
determine a root code associated with the identified ontology code;
obtain, from a database, child codes associated with the root code;
transmit, to a large language machine (LLM) engine, the at least portion of text from the document, the obtained child codes, and a prompt instructing the LLM engine to determine one or more specific ontology codes based on the at least portion of the text utilizing the obtained child codes; and
receive, from the LLM engine output that includes one or more specific ontology code.

14. The computer readable medium according to claim 13, wherein the instructions, when executed by the at least one processor, further cause the processor to:
generate, utilizing a fine-tuned language model engine, structured data based on the at least portion of text from the document by:
identify, in the at least portion of text, a span of text;
identify a concept that is associated with text included in the identified span of text; and
wherein the structured data includes the identified concept and an identification of the identified span of text associated with the identified concept;
wherein the structured data is transmitted to the LLM engine with the at least portion of text from the document, the obtained child codes, and the prompt, wherein the prompt instructs the LLM engine to determine the specific ontology code for the at least portion of the text utilizing the structured data.

15. The computer readable medium of claim 13, wherein the instructions that, when executed by the at least one processor, cause the processor to obtain child codes associated with the root code comprise instructions that, when executed by the at least one processor, cause the processor to perform a wildcard search for child codes that match the root code.

16. The computer readable medium of claim 13, wherein:
the prompt further instructs the LLM engine to generate reasons for each of the determined one or more specific ontology codes; and
the instructions that, when executed by the at least one processor, cause the processor to receive the output from the LLM engine comprise instructions that, when executed by the at least one processor, cause the processor to receive, for each of the determined one or more specific ontology codes, the generated reasons.

17. The computer readable medium of claim 13, wherein the document is a medical records document, and the context-specific NLP is specific to the medical field.

18. The computer readable medium of claim 17, wherein the identified and specific ontology codes comprise ontology codes set forth in the International Classification of Diseases version 10 Clinical Modification (ICD-10), and the database is a database that includes the ICD-10 codes.
